(19) United States
(12) Patent Application Publication
(10) Pub. No.: US 2021/0094105 A1
(43) Pub. Date: Apr. 1, 2021

(54) TECHNOSCOPIC INSTRUMENT

(71) Applicant: Richard Wolf GmbH, Knittlingen (DE)

(72) Inventor: Daniel SCHWAB, Knittlingen-Freudenstein (DE)

(21) Appl. No.: 17/039,135

(22) Filed: Sep. 30, 2020

(30) Foreign Application Priority Data
Oct. 1, 2019 (DE) 20 2019 105 420.4

(51) Int. Cl. B23B 45/00; B24B 27/027

(52) U.S. Cl. CPC B23B 45/005; B23Q 17/24; B24B 27/027

(57) ABSTRACT

A technoscopic instrument (1) for machining surfaces in technical cavities, includes a stationary, tubular shank (7) and a tool unit (11), for holding a machining tool (13), arranged distally on the shank (7) and pivotable transversely to a longitudinal axis (37) thereof by way of a joint (9). The tool unit (11) is coupled to a first adjusting unit (17) which is arranged proximally on the instrument (1). The pivotable tool unit (11) includes an end section (43) which is arranged distally to the joint (9), is mounted in an axially displaceably and via an elongate control element (49) which is pull-rigid, push-stiff and pliable transverse to its longitudinal axial extension is coupled to a second adjusting unit (23) which is arranged proximally on the instrument (1) and which is provided independently and separately of the first adjusting unit (17), for selective telescoping of the end section (43).

### CROSS REFERENCE TO RELATED APPLICATIONS

This application claims the benefit of priority under 35 U.S.C. § 119 of German Application 20 2019 105 420.4, filed Oct. 1, 2019, the entire contents of which are incorporated herein by reference.

### TECHNICAL FIELD

The disclosure relates to a technoscopic instrument for machining surfaces in technical cavities, wherein the instrument comprises a stationary, tubular shank and a tool unit for receiving a machining tool, said tool unit being arranged distally on the shank and being pivotable transversely to a longitudinal axis of the shank by way of a joint, wherein the tool unit, for the selective pivoting is coupled to a first adjusting unit which is arranged proximally on the instrument.

### TECHNICAL BACKGROUND

Such a technoscopic instrument can be used for carrying out repair work on components in technical systems, without a laborious disassembly of the respective components having to be effected. For this, the tool unit and the shank can be inserted through a suitable opening into the respective system. During the operation of the instrument, a user can observe a picture which is acquired by optics, wherein these optics run out directly on or adjacent to a distal end of the tool unit. The tool unit can carry different types of machining tools, in particular rotating tools such as drills, millers, grinders and the like.

In such technical cavities, the most varied of demands on the machining can be placed upon the technoscopic instrument, such demands possibly necessitating different tools. Depending on the ascertained state of the surface to be machined, in particular some regions can be reached better with a first tool than other regions which would demand a differently shaped second tool. However, it is laborious and time-consuming to successively equip the tool unit with different tools. Furthermore, the provision of several different machining tools for an application as is required increases the entailed investment for the technoscope instrument.

### SUMMARY

An object of the invention lies in the improved use of a single tool in comparison to known technoscopic instruments of this type. In particular, the improved use is to expand a working field of the tool instrument without restricting the operating ability or flexibility of the instrument.

A technoscopic instrument for machining surfaces in technical cavities is put forward, wherein the instrument comprises a stationary, tubular shank and a tool unit for holding a machining tool, said tool unit being arranged distally on the shank and being pivotable transversely to a longitudinal axis of the shank by way of a joint, wherein the tool unit, for the selective pivoting is coupled to a first adjusting unit which is arranged proximally on the instrument. According to the invention, the pivotable tool unit comprises an end section which is arranged distally to the joint, is mounted in an axially displaceable manner and via an elongate control element which is pull-rigid, push-stiff and pliable transverse to its longitudinally axial extension is coupled to a second adjusting unit which is arranged proximally on the instrument and is provided independently and separately of the first adjusting unit for the selective telescoping of the end section.

Concerning the technoscopic instrument according to the invention, as a result, additionally to the pivoting ability of the tool unit, a simple telescoping ability which is realized independently of this is provided. Consequently, the machining tool which is attached to the tool unit can be moved relative to the tool unit in the axial direction, so that this is axially lengthened or shorted according to requirements. The area or surface which can be reached by the machining tool by way of the targeted operation of the two independent adjusting units is significantly increased by way of this, in comparison to known technoscopic instruments. A laborious exchange of machining tools in order to enlarge the field of application is however not necessary for the majority of applications.

The end section of the tool unit is to be understood as a proximal, axial section of the tool unit and is achieved by way of an axial subdivision. The end section for example could be guided axially on a pivoting section which is connected to the joint or comprises this. By way of this, the end section can be moved in the axial direction relative to the pivoting section.

The axial movement is herein achieved by the elongate control element which is coupled to the second adjusting unit. The first adjusting unit and the second adjusting unit are operable separately from one another and can initiate the pivoting and telescoping independently of one another.

The elongate control element runs from the second adjusting unit to the end section of the tool unit and herewith runs through the joint. On account of its pliable configuration, the control element can follow a direction change on the joint which is caused by the pivoting, without further ado. Since the control element is moreover pull-rigid and push-stiff, a force which is exerted upon the control element leads to a targeted movement of the end section. One can envisage the control element being configured in particular as a wire or a wire-like element of a non-metallic material and serving as a pull means as well as a push means. On the one hand, it could be flexible enough, in order to follow the pivoted tool unit, but on the other hand sufficiently stiff, in order not to abruptly bend given its use as a push means.

Optionally, the control element comprises at least one wire. The control element could partly, largely completely or completely consist of one wire or of several wires. Depending on the distance which is to be bridged between the joint and a completely extended position of the end section, a suitable diameter of the wire results, in order to simultaneously achieve the necessary push-stiffness and despite this to permit a bending of the control element for following the alignment of the end section. In particular, the wire can be a stainless-steel wire or comprise other materials. The at least one wire can be also be configured as a braid or stranded wire with several wires which encircle one another. Furthermore, it is conceivable for at least one axial part-section to be protected by a sheathing, wherein the sheathing could be flexurally rigid and counteract a bending in an envisaged region of the instrument.

Further optionally, the tool unit can comprise a sleeve section and a cylinder section which is axially displaceable therein. The sleeve section is part of the tool unit and has the shape of a sleeve. The sleeve section could be provided on the side of the tool unit which is away from the joint or be provided distally and hence realize the end section. The cylinder section has a shape which corresponds to the sleeve section and can be stuck into an open end of the sleeve section, in order to be led there in the axial direction. If the cylinder section is located distally on the tool unit, then this can represent the end section.

In an advantageous embodiment, the sleeve section could be coupled to the joint, wherein the cylinder section as an end section is connected to the control element. It is axially displaced by the control element and by way of this determines the axial position of the machining tool. The cylinder section at a proximal end can comprise fastening means for fastening the control element. The fastening means amongst other things could comprise a bore, into which the control element is led. There, it could be for example bonded, welded or clamped.

Furthermore, the control element could be distanced to a longitudinal axis of the shank and in the tool unit assumes a coaxial alignment with the tool unit. The control element consequently runs eccentrically to a longitudinal axis of the shank, in particular in the inside of the shank and by way of this can be led past the joint at the peripheral side in a simple manner. Since the end section of the tool unit is to be displaced axially, a middle concentric position of the control element on the tool unit is particularly favorable, in order to reliably prevent a jamming given the axial movement.

The tool unit could comprise a run-in region for the control element, said run-in region facing the joint and tapering in the distal direction. In this run-in region, the control element runs from the proximal direction into the tool unit. Since a corresponding bending of the control element is always effected due to the pivoting of the tool unit, the control element can assume a running direction relative to the longitudinal extension of the tool unit, which is dependent on a pivoting of the tool unit. A funnel-like run-in region which simplifies a direction adaptation of the control element after passing the joint results due to an opening which widens towards the joint or which tapers in the distal direction.

Furthermore, the shank in a region which is adjacent to the joint could comprise at least one guide element which is distanced to the tool unit and which limits a sagging of the control element in at least one direction. The guide element in a certain pivoting range of the tool unit generally permits an unhindered bending of the control element. However, in order to prevent a loss of the transmission of a push force and/or a damage to the control element, an intensity of the sagging can be limited by way of a suitable placement of the at least one guide element. It is further conceivable to provide two or more guide elements which limit the sagging in two different directions. These for instance could lie opposite one another.

Here, it should be noted that the control element for further limiting its sagging could be guided through a gap which runs in the instrument and in particular on the joint and has an extensive extension with main extension planes transverse to a hinge axis of the joint. The thickness of the gap would then be selected somewhat larger than the respective cross-sectional measure of the control element, so that its unhindered movement is possible.

For pivoting, the tool unit can optionally be coupled eccentrically to a push rod which is led eccentrically in the shank and which is pivotable by way of the first adjusting unit. The push rod can furthermore likewise be realized by a partly flexible or pliable element which is provided for example at least partly with a sheathing. The use of a push rod also permits the transmission of larger forces, in order to be able to accommodate forces which occur transversely to the tool unit, in particular counter-forces which arise on machining at the machining tool.

It is advantageous if the joint or the shank comprises at least one stop surface for limiting a pivoting angle of the tool unit. The stop surface is configured to assume a surface contact with the tool unit in certain pivot positions. By way of this, a mechanical limitation of the pivot angle is achieved, which makes the operation of the instrument simpler for the user by way of a clear mechanical feedback of the reached end position be effected on operating the first adjusting unit. Furthermore, one prevents an excessive pivoting and unintended contact of the machining surface with surfaces of the technical cavity. Hence, an undesirable abrupt bending can be prevented by way of preventing an excessive bending of the control element.

The tool unit is particularly advantageously pivotable by up to 120°. This pivoting range is sufficiently large, in order to permit a significant enlargement of the operating region. The control element can easily follow the tool unit over such a pivoting range without the danger of an abrupt bending arising. If the control element is to be arranged in a gap, this gap could furthermore be dimensioned in a manner such that a harmonic course of the control element in the gap is possible, such a course being suitable for the loading, and a straining or rubbing of the control element is prevented.

Particularly preferably, the end section is displaceable in a rotationally fixed manner. A transmission of torque for driving a machining tool and the accommodation of counter-forces on operation of a machining tool with a pivoted tool unit is possible by way of this without further measures.

The invention is hereinafter explained in more detail by way of embodiment examples which are represented in the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a lateral view of a technoscopic instrument;

FIG. 2a is a lateral view showing a non-pivoted tool unit;

FIG. 2b is a lateral view showing a pivoted tool unit;

FIG. 3a is a lateral view with a telescoped end section;

FIG. 3b is a partly sectioned lateral view with a non-telescoped end section;

FIG. 3c is a partly sectioned lateral view with a telescoped end section; and

FIG. 4 is a detailed sectioned representation of a joint.

### DESCRIPTION OF PREFERRED EMBODIMENTS

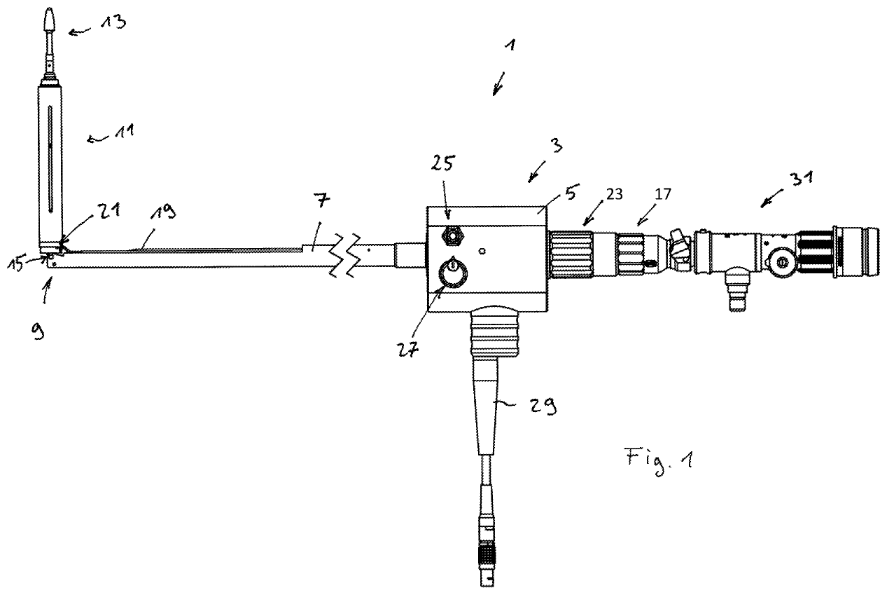

Referring to the drawings, FIG. 1 shows a lateral view of a technoscopic instrument 1 for machining surfaces in technical cavities. The instrument 1 comprises a hand grip 3 with a housing 5, on which a stationary, tubular shank 7 is arranged. A joint 9 which carries a tool unit 11 is arranged at a distal end of the shank 7. This represents a distal lengthening of the shank 7 and is configured to hold the machining tools 13. The joint 9 comprises at least one hinge axis 15 which runs transversely to the shank 7, for achieving a pivotability.

A first adjusting unit 17 is arranged on an end of the housing 5 which is opposite to the shank 7 and is configured by way of example as an adjusting wheel with a peripheral-side knurling. The first adjusting unit 17 by way of example is connected to a push rod 19 which runs eccentrically in the shank 7 in the direction of the joint 9. There it is coupled to the tool unit 11 at an eccentrically arranged pivoting point 21 which is close to the joint 9. The push rod 19 can be displaceably mounted and can be arranged merely running freely in the shank 7. The first adjusting unit 17 is formed by a mechanism which is not shown here and which is capable of moving the push rod 19 along the shank 7 in a manner such that the tool unit 11 is pivoted about the hinge axis 15.

A second adjusting unit 23 which is arranged distally adjacent to the first adjusting unit 17 is likewise configured exemplarily as an adjusting wheel with a peripheral knurling. In the shown variant, the first adjusting unit 17 and the second adjusting unit 23 are configured coaxially to one another and arranged axially one after the other. The second adjusting unit 23, as is explained hereinafter, is provided in order to achieve an axial displacement of the machining tool 13.

By way of example, a switch 25 for switching an operation of the machining tool 13 on or off is located on an outer side of the housing 5. This could be configured in a rotating manner. A speed of the machining tool 13, by way of example could be controlled via a speed controller 27 which is likewise arranged on the housing 5. A supply conduit 29 which for example serves for the electricity supply of a drive of the machining tool 13 connects to an end which is at the bottom in the plane of the drawing. It is conceivable for the machining tool 13 to be provided with an electric motor which in the representations is not shown in detail for the sake of simplicity and which could be placed either directly on the distal end of the tool unit 11 or within the housing 5. Finally, a rigid endoscope 31 is located at a proximal end of the instrument 1 and with the help of suitable optics permits an observation of the operating region by a user with the aid of suitable optics. The optics could herein run to into the tool unit.

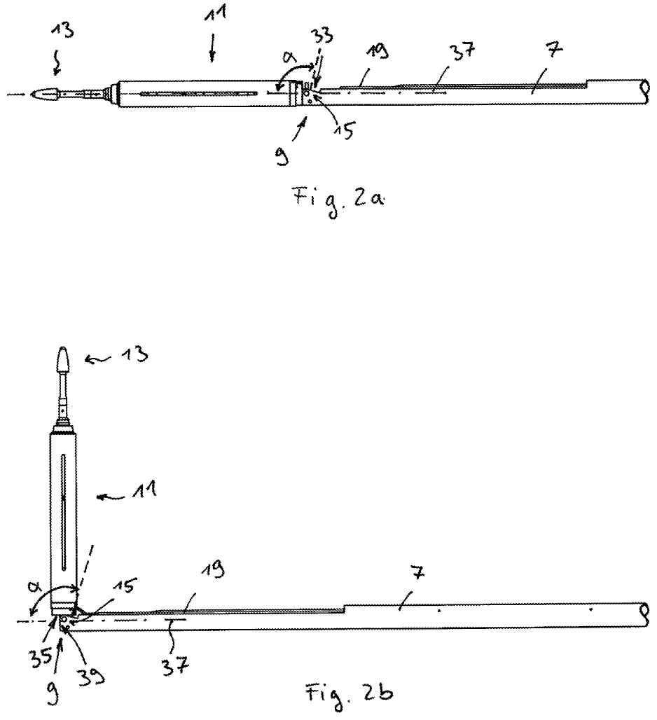

In FIGS. 2a and 2b, the pivoting of the tool unit 11 is represented in two lateral views. FIG. 2a illustrates the tool unit 11 in a non-pivoted position, in which the stationary shank 7 and the tool unit 11 are arranged concentrically to one another. The push rod 19 is located in a maximally extended distal position and the pivoting point 21 is located in its distal end position. In FIG. 2b, the tool unit 11 is shown in an approximately maximally pivoted position.

The shank 7 in the proximity of the joint 9 comprises a first stop surface 33 which is arranged laterally on the shank 7 and which is configured to come into a surfaced contact with a proximal end surface 35 of the tool unit 11. By way of this, a possible pivoting range of the tool unit 11 is limited to an angular range a which is set by the first stop surface 33. This for example could encompass a range of up to 120° and lie between the surface normals on the first stop surface 33 and a longitudinal axis 37 of the shaft 7. The straight-lined concentric alignment of the tool unit 11 to the shank 7, which is shown in FIG. 2a, further corresponds with the second stop surface 39 which here is aligned perpendicularly to the longitudinal axis 37.

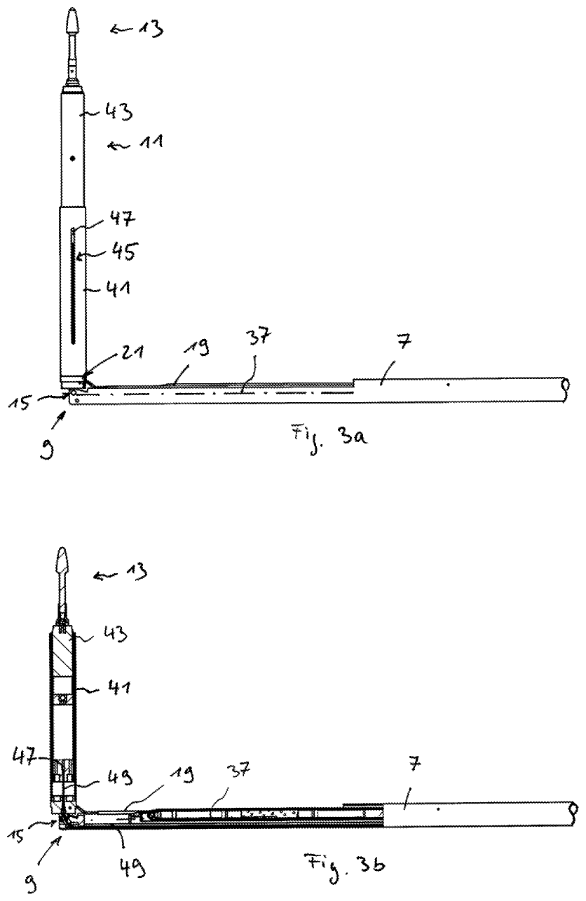

As is represented in the FIGS. 3a to 3c, the tool unit 11 can be axially telescoped, so that the machining tool 13 is axially displaced. In FIG. 3a, the tool unit 11 is situated in an extended position. For this, the tool unit 11 comprises a sleeve section 41 which is mounted on a joint 9 and, as an end section, a cylinder section 43 which is axially displaceable therein and which determines the distal extension of the tool unit 11. The cylinder section 43 has an outer diameter which is slightly smaller than the inner diameter of the sleeve section 41 which surrounds it. A simple but robust mechanical guidance results by way of this. In order to prevent a rotation of the cylinder section 43, the sleeve section 41 further comprises at least one axially running slot 45, in which a transverse pin 47 of the cylinder section 43 is led. It is conceivable for the sleeve section 41 to comprise such a slot 45 at two sides which are opposite one another, through which slot a single or several transverse pins 47 extend. The radius of action of the machining tool 13 is significantly enlarged due to the displacement of the cylinder section 43.

For moving the cylinder section 43, this is coupled to an elongate control element 49 which is led eccentrically in the stationary shank 7 and is movable in a longitudinal-axial manner by way of the first adjusting element 17. The control element 49 is pliable transverse to its longitudinal axis, but is pull-rigid and push-stiff (tensionally rigid and compressively stiff). It can be configured similarly to a Bowden cable at least in sections. The control element 49 on account of the pliable character is in the position of being led past the joint 9 over the complete pivoting range. If the tool unit 11 is pivoted, then the control unit 49 can very easily follow this movement. If it is moved in a longitudinal-axial manner, then the cylinder section 43 can follow this movement. One account of the tensionally and compressionally rigid characteristics, the cylinder section 43 can be retracted as well as extended.

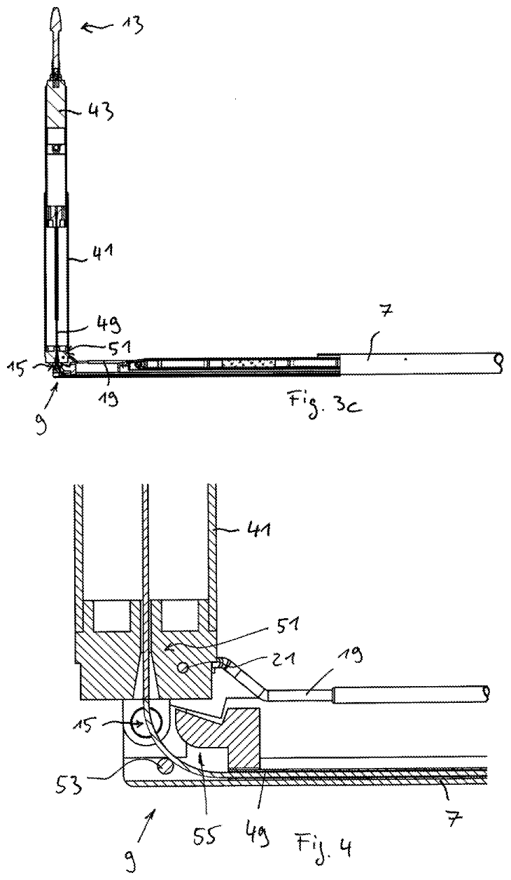

FIG. 3c by way of example shows the extended position of the tool unit 11, at which the control element 49 is completely pushed out. On account of the pliable characteristics, the control element 49 however is not in the position of being able to transmit push forces over an unlimited stretch, without it being restricted in its movability in the transverse direction. For this, guide elements are applied in the shown representation and these are clear by way of FIG. 4 and limit a sagging of the control element 49.

FIG. 4 in a sectioned detailed view shows the course of the control element 49 in the region of the joint 9. The sleeve section 41 for guiding the control element 49 comprises a run-in region 51 which tapers in the distal direction. By way of this, the region of the run-in region 51 which faces the joint 9 is configured in a roughly funnel-like manner. By way of example, the run-in region 51 is arranged concentrically in the tool unit 11 and leads the control element 49 centrally onto the cylinder section, so that given its movement the damage of a jamming is minimized. By way of the run-in region 51, the control element 49 can also follow differently pivoted tool units 11 without it rubbing along a sharp-edged opening.

For leading the control element 49 for limiting a sagging, the shaft 7 by way of example can comprise a first guide element 53 which is positioned in a region which is adjacent to the joint 9, and is distanced to the tool unit 11. In a simple case, the first guide element 53 can be configured as a pin which runs transversely to the longitude axis 37 of the shank 7 and in at least one angular range of the tool unit 11 can come into a surfaced contact with the control element 49. A sagging which goes beyond this is then reliably prevented.

Furthermore, a second guide element 55 which transversely to the longitudinally axial extension of the control element 49 is distanced to the first guide element 53 and which is offset proximally somewhat is provided. By way of example, the second guide element 55 comprises a convex surface which can likewise come into a surface contact with the control element 49. The control element 49 is then always located between the two guide elements 53 and 55 and is then distanced at least from one of the two guide elements 53 and 55. If the tool unit 11 is brought into a coaxial position with the shank 7, the control element 49 snuggles onto the second guide element 55 so that its sagging is limited and the control element 49 can continue to transmit pulling and pushing forces. Given a high pivoting angle, the control element 49 again comes into surface contact with the first guide element 53.

The pivoting point 21 which is coupled to the push rod 19 is also shown in more detail in FIG. 4. The push rod 19 could be coupled to the pivoting point 21 via a joint fork or merely comprise a bent or angled end which is stuck into the tool unit 11. The articulated connection leads to a tilting movement of the tool unit 11 about the joint 9 or the hinge axis 15 which is defined by this.

While specific embodiments of the invention have been shown and described in detail to illustrate the application of the principles of the invention, it will be understood that the invention may be embodied otherwise without departing from such principles.

### LIST OF REFERENCE NUMERALS

1 technoscopic instrument
3 hand grip
5 housing
7 stationary shank
9 joint
11 tool unit
13 machining tool
15 hinge axis
17 first adjusting unit
19 push rod
21 pivoting point
23 second adjusting unit
25 switch
27 speed controller
29 supply conduit
31 endoscope
33 first stop surface
35 proximal end surface
37 longitudinal axis
39 second stop surface
41 sleeve section
43 cylinder section, end section
45 slot
47 transverse pin
49 control element
51 run-in region
53 first guide element
55 second guide element

What is claimed is:

1. A technoscopic instrument for machining surfaces in technical cavities, the technoscopic instrument comprising:
a stationary, tubular shank;
a pivotable tool unit for holding a machining tool, said tool unit being arranged distally on the shank and being pivotable transversely to a longitudinal axis of the shank by way of a joint, wherein the pivotable tool unit is coupled to a first adjusting unit which is arranged proximally on the technoscopic instrument, wherein the pivotable tool unit comprises an end section arranged distally to the joint, the end section being mounted axially displaceably; and
an elongate control element which is pull-rigid, push-stiff and pliable transverse to a control element longitudinal axial extension, the elongate control element being coupled to a second adjusting unit which is arranged proximally on the technoscopic instrument and which is provided independently and separately of the first adjusting unit, for a selective telescoping of the end section.

2. A technoscopic instrument according to claim 1, wherein the elongate control element comprises at least one wire.

3. A technoscopic instrument according to claim 1, wherein the pivotable tool unit comprises a sleeve section and a cylinder section which is axially displaceable in the sleeve section.

4. A technoscopic instrument according to claim 3, wherein the sleeve section is coupled to the joint, and the cylinder section as an end section is connected to the elongate control element.

5. A technoscopic instrument according to claim 1, wherein:
the elongate control element is spaced a distance relative to a longitudinal axis of the shank; and
in pivotable tool unit the elongate control element assumes a coaxial alignment with pivotable tool unit.

6. A technoscopic instrument according to claim 5, wherein:
pivotable tool unit comprises a run-in region for the elongate control element;
said run-in region faces the joint and tapers in a distal direction.

7. A technoscopic instrument according to claim 1, wherein the shank in a region which is adjacent to the joint comprise at least one guide element which is spaced a distance to the pivotable tool unit and which limits a sagging of the elongate control element in at least one direction.

8. A technoscopic instrument according to claim 1, further comprising a push rod, wherein for pivoting, the pivotable tool unit is coupled eccentrically to the push rod and the push rod is led eccentrically in the shank and is pivotable by way of the first adjusting unit.

9. A technoscopic instrument according to claim 1, wherein the joint or the shank comprises at least one stop surface for limiting a pivoting angle of pivotable tool unit.

10. A technoscopic instrument according to claim 1, wherein pivotable tool unit is pivotable by up to 120°.

11. A technoscopic instrument according to claim 1, wherein the end section is rotationally fixedly displaceable.
